(19) United States
(12) Patent Application Publication
(10) Pub. No.: US 2014/0274920 A1
(43) Pub. Date: Sep. 18, 2014

(54) METHODS OF TREATING OR PREVENTING OVERWEIGHT AND OBSESITY IN MAMMALS BY ADMINISTERING A COMPOSITION COMPRISING MANNOHEPTULOSE

(71) Applicant: The Iams Company, Cincinnati, OH (US)

(72) Inventor: Gary Mitchell Davenport, Dayton, OH (US)

(73) Assignee: The Iams Company, Cincinnati, OH (US)

(21) Appl. No.: 14/198,680

(22) Filed: Mar. 6, 2014

Related U.S. Application Data
(60) Provisional application No. 61/786,557, filed on Mar. 15, 2013.

(51) Int. Cl. A61K 36/54; A61K 36/60; A61K 36/185; A61K 36/48

(52) U.S. Cl. CPC A61K 36/54; A61K 36/48; A61K 36/60; A61K 36/185
USPC 514/23

(57) ABSTRACT

The present invention is based on the discovery that mannoheptulose (mHep) reduces or inhibits the differentiation process of fat stem cells into adipocytes so as to decrease the body's fat storage capacity. Compositions comprising a mHep, process of making the compositions, and methods of using the compositions for treating or preventing overweight and obesity in a mammal, such as, for example, a growing pet (e.g., puppy, kitten), or a spayed/neutered pet, are encompassed by the present invention.

This application claims the benefit of U.S. Provisional Application Ser. No. 61/786,557, filed Mar. 15, 2013, the entirety of which is incorporated by reference herein.

### FIELD OF THE INVENTION

The present invention is directed to a method of treating or preventing overweight and obesity in a mammal comprising administering to the mammal a composition comprising an effective amount of a mannoheptulose (mHep). Further, the present invention encompasses a composition comprising a mHep and a process of making the composition.

### BACKGROUND OF THE INVENTION

Overweight and obesity are significant problems in mammals, particularly in humans and domesticated animals such as, for example, pets (e.g., dogs, cats). According to the United States Centre for Disease Control (CDC), about ⅓ (33.3%) of all adult humans in the U.S. are overweight (i.e., BMI=25 kg/m2 to 29.9 kg/m2), and more than ⅓ (35.7%) are obese (i.e., BMI>30 kg/m2). Unfortunately, the pets are not any healthier than their owners. According to the 2011 statistical data from the Association for Pet Obesity Prevention's (APOP), in the United States 53% of adult dogs and 55% of adult cats are either overweight or obese (based on similar scoring system and the Body Condition Chart (BCS) for Assessing Obesity in Cats and Dogs, developed by Greg Sunvold from IAMS® (1998)).

It is important to note that whether humans and domesticated animals are considered overweight or obese does not depend on body weight, but instead on the percentage of body fat. Overweight and obesity is the accumulation of excess adipose tissue (i.e., body fat) due to, for example, overconsumption of calories and/or lack of exercise. Excessive accumulation of body fat occurs when the synthesis and deposition of fat (e.g., triglycerides) into adipocytes exceed their breakdown. Over time, this excessive accumulation of body fat leads to overweight then obesity, if left untreated.

Being overweight or obese will clearly increase morbidity and mortality. It may also exacerbate many health problems, both independently and in conjunction with other diseases. For example, if overweight or obese dogs are left untreated, especially as adult dogs (i.e., about 6 to 8 years old) or senior dogs (i.e., about 9 years old and above), then their quality of life may decrease due to the increased risks of developing chronic diseases such as, for example, diabetes mellitus, cancer, hypertension, pulmonary cardiovascular, or degenerative joint disease. Thus, the benefits of not being overweight or obese include not only a longer life, but also provide a higher quality of life. Senior dogs are further disadvantaged due to the fact that they are susceptible to “age-related increase in body fat” that may contribute to becoming overweight or obese. Accordingly, the need still exists for new or improved methods of treating or preventing overweight and obesity in mammals, particularly where the mammal is a senior pet.

In addition to adipose tissue's role as a repository of surplus energy (i.e., fat) for the body, it has been recognized as an endocrine organ that produces hormones such as, for example, the sex hormone estrogen. Estrogen is present in both male and female animals. It plays an important role in regulating the function and development of adipocytes that form the adipose tissue and maintenance of normal body fat composition (Cooke, P. S. et al., Exp. Biol. Med., 229:1127-35, 2004). In fact, diminished estrogen levels, for example, after menopause in humans and the spaying/neutering of pets for population control reasons, have been correlated with increased accumulation of adipose tissue (body fat) (Pergola, G. D., Int. J. Obesity, 24:S59-S63, 2000; Cooke, P. S. et al., (2004), supra.; and Mohamed, M. K., et al., Eur. J. Endocrinol. 15 142:307-14, 2000).

Estrogen may mediate its effects on adipose tissue in different ways. For example, estrogen may affect the number and size of adipocytes; modulate the rate of lipogenesis and lipolysis; and increase appetite or decrease energy expenditure (Pergola, G. D., (2000), supra; Cooke, P. S. et al., (2004), supra; and Naaz, A., et al., Endocrinology 144:3315-20, 2003). Hormone replacement therapy and dietary supplementation with estrogen are common approaches that attempt to reverse these effects (Sayegh, R. A., et al., Meopause 6:312-5, 2002). However, both therapies may be expensive and require strict patient compliance with the regimens in order for them to be effective.

Accordingly, the need still exists for new or improved methods of treating or preventing overweight or obesity in a mammal, which may result from weight gain due to, for example, menopause in humans or spaying/neutering of animals, particularly wherein the methods require minimal additional cost and are easy for the patient/animal to comply. For example, the mHep is already incorporated in the pet food composition, then the animal only needs to eat to be treated.

Consumers believe healthy longevity of their pet can be achieved through a combination of proper nutrition from a high-quality diet, spaying and neutering, maintenance of an active lifestyle, and routine visits to veterinary. The pet food industry has explored the use of caloric restriction mimetic as a strategy for delivering against this demand of proper nutrition in pet food. It is believed that caloric restriction leads to an increase in median lifespan (i.e., age at 50% population survival) and maximal lifespan (i.e., age of longest lived individual). Recent studies show that the amount of time the lifespan of an animal is extended will progressively increase as caloric in-take is reduced and the duration of the caloric restricted diet increases. In one study, the lifespan of a monkey was increased by more than 30% because 5 the animal had started on caloric restriction shortly after birth (Mattson, M. P. et al., J. Nutr. Biol. 16, 3:129-137, 2005). It has also been shown that 10% to 40% reductions in caloric in-take are effective to improve the quality of life by delaying and/or preventing age-related diseases.

An example of a caloric restriction mimetic is mannoheptulose (mHep), which is a seven carbon sugar found in a variety of natural sources such as, for example, avocados, figs, and primrose. It is believed that mHep is effective for extending lifespan and life quality in both lower and higher organisms. As a caloric-restriction mimetic, mHep competes with glucose for binding to hexokinase-1, an enzyme in the glycolysis reaction, due to the similarity between their structures. The result is that glucose metabolism is inhibited and fatty acid synthesis is prevented. This leads to decrease levels of fat deposition in adipocytes that may eventually lead to weight loss. In a related role, mHep may function as a glucose anti-metabolite to block the normal metabolism of carbohydrate. The resulting physiological changes may include: lower tissue glucose levels, decrease plasma insulin levels and reduction of body weight (see PCT Publication No. WO2012/61446A1). Both effects result from mHep's inhibition of carbohydrate 20 metabolism. To date, mHep has not been shown to impact adipocyte expression or activity, and particularly not linked to overweight and obesity.

Given the magnitude of the growing problem with excessive weight gain in today's human and pet populations, there is still a need for new or improved methods of treating and preventing overweight and obesity in mammals. In an embodiment, the mammals are preferably chosen from sub-groups that have elevated risk of adiposity brought on by, for example, changes in hormonal homeostasis (i.e., menopausal humans and spayed/neutered pets) or arose from normal age-related weight gains (i.e., senior humans or senior pets), particular if the animal is otherwise healthy. It is desirable that the new or improved methods would also benefit growing pets, preferably if the growing pets are overweight or obese or have been spayed/neutered before reaching sexual maturity. Alternatively, by administering mHep early-on to the growing pet, the animal is exposed to caloric restriction mimetic for a longer duration thereby increasing the chance of extending the lifespan in those animals.

### SUMMARY OF THE INVENTION

In a first aspect, the present invention is directed to a new use of mHep based on the discovery of different molecular processes and mechanisms by which mHep provides new benefits to the mammal. The inventor discovered that mHep inhibits fat 5 stem cells from differentiation into adipocytes, and thereby lessens the body's fat storage capacity due to reduced levels of adipocytes. It has also been discovered that mHep inhibits adipocytes from exhibiting adipogenic or lipogenic effect that may result in the excessive build-up of fatty tissue contributing to body weight increase. By this discovery, the inventor have identified new methods of using mHep for treating or preventing overweight and obesity in mammals, wherein the method comprises administering to the mammal a composition comprising an effective amount of a mHep.

In one embodiment, the method relates to a mammal that is a pet, more particularly, wherein the pet is selected from: (a) a growing pet (e.g., puppy, kitten) that may be overweight or obese, reproductively-intact, or spayed/neutered; (b) an adult pet that may be overweight or obese, reproductively-intact, or spayed/neutered; or (c) a senior pet that may be overweight or obese, reproductively-intact, or spayed/neutered.

In another embodiment, the method comprises administering to the mammal a composition comprising an effective amount of an mHep to inhibit or reduce the differentiation process of fat stem cells into adipocytes in the mammal such that the mammal's body fat storage capacity decreases by about at least 5%, about at least 10%, or about at least 20%, relative to an untreated mammal.

In yet another embodiment, the method wherein the administration comprises feeding to the mammal a composition comprising a daily dosage amount of from about 1 mg/kg to about 25 mg/kg of a mHep, and wherein the composition is selected from the group consisting of a pet food, a supplement, and a liquid, wherein the supplement or the liquid can be taken separately or added to the pet food. The composition can be selected from the group consisting of wet composition (i.e., having a total moisture content of greater than 50%, by weight of the product), moist composition (i.e., having a total moisture content of from about 16% to about 50%, by 30 weight of the product), semi-moist composition, dry composition and combinations thereof. The composition can be nutrionally balanced pet food composition.

In another aspect, the present invention is directed to a method of increasing the lifespan of a mammal, preferably a growing pet such as, for example, a puppy or a kitten, by administering to the mammal a composition comprising an effective amount of a mHep to reduce the caloric in-take of the young pet, wherein the administration starts within a window of about one month to 3 years of the birth of the animal. The reduction in caloric in-take is preferably anywhere within a range of about 10% to about 50% of the caloric in-take prior to the administration.

In yet another aspect, the present invention is directed to a pet food composition comprising an effective amount of an mHep for promoting or maintaining a healthy body fat of growing pets, and wherein the mHep is present in the composition at from about 0.0001% to about 10% by weight of the composition. The pet food composition is preferably a high fat food composition with a fat content present in the composition from at least about 15% to at least about 45% by weight of the composition. The higher fat content provides more palatability to the pet food composition, while the mHep minimizes and/or counters any negative effects on body weight gain.

These and other features of the present invention will become apparent to one skilled in the art upon review of the following detailed description when taken in conjunction with the appended claims.

### BRIEF DESCRIPTION OF THE DRAWINGS

While the specification concludes with claims particularly pointing out and distinctly claiming the invention, it is believed that the invention will be better understood from the following description of the accompanying figures wherein:

FIG. 1A shows the body condition chart for assessing obesity in dogs.

FIG. 1B shows the body condition chart for assessing obesity in cats.

FIG. 2 shows the oil staining showing mHep inhibiting MSC differentiation.

### DETAILED DESCRIPTION OF THE INVENTION

### Definitions

As used herein, articles such as “a” and “an” when used in a claim, are understood to mean one or more of what is claimed or described. Within the context of this specification the term “about” is interpreted to mean optionally plus or minus 20%, preferably optionally plus or minus 10%, more preferably optionally plus or minus 5%, even more preferably optionally plus or minus 2%, and most preferably optionally plus or minus 1%.

As used herein, the terms “include”, “includes” and “including” are meant to be nonlimiting. “Obesity”, as used herein refers to an increase in body weight beyond the limitation of skeletal and physical requirement, as a result of excessive body fat accumulation. A human subject is “obese” if he has a Body Mass Index (BMI) above 30 kg/m2 according to the United States CDC guidelines. A pet such as, for example, a cat or dog, is identified as being “obese” if the animal possess the physical attributes using the guidelines set out in the Body Condition Chart (BCS) for Assessing Obesity in Cats and Dogs, developed by Greg Sunvold from IAMS® (1998), and reproduced in FIGS. 1A (dogs) and 1B (cats). For all other types of mammals, similar Body Condition Charts and scoring systems, such as a Body Condition Score, are available to provide guidance for determining whether the subject is “obese”.

“Overweight” as used herein means weighing more than is normal or necessary, especially having more body weight than is considered normal or healthy for one's age or build. A human subject is “overweight” if he has a Body Mass Index (BMI) between 25.0 kg/m2 to 29.9 kg/m2 according to the United States CDC guidelines. A pet such as, for example, a cat or dog, is identified as being “overweight” if the animal possess the physical attributes using the guidelines set out in the Body Condition Chart (BCS) for Assessing Obesity in Cats and Dogs, developed by Greg Sunvold from IAMS® (1998), and reproduced in FIGS. 1A (dogs) and 1B (cats). For all other types of mammals, similar Body Condition Charts and scoring systems are available to provide guidance for determining whether the subject is “obese”. “Mammal” as used herein means a primate, such as a human, ape or monkey, or domesticated animal such as, for example, a pet.

“Pet” as used herein means a domesticated animal including, domesticated dogs (canines), cats (felines), horses, cows, ferrets, rabbits, pigs, rats, mice, gerbils, hamsters, horses, and the like. Pet is also referred to as “companion animals” herein.

“Growing pet” as used herein refers to a subset of “pet” and includes young animals that are still undergoing development and are not yet sexually mature.

“Kitten” as used herein refers to a type of “growing pet” and includes domesticated cats which are 3 years old or less, or about 2 years old or less, or about 1 year old or less.

“Puppy” as used herein refers to a type of “growing pet” and includes domesticated dogs which are 3 years old or less, or about 2 years old or less, or about 1 year old or less.

“Adult pet” as used herein refers to a subset of “pet” and includes, for example, domesticated dogs (canines) and cats (felines) that are between about 3 years old and about 8 years old.

“Senior pet” as used herein refers to a subset of “pet” and includes, for example, domesticated dogs (canines) and cats (felines) that are about 9 years old and above. “Reproductively-intact” as used herein means that the animal can still reproduce as they have not undergone any procedures to remove or disable the sexual organs (e.g., ovaries, testicles).

“Menopause” as used herein means the period of permanent cessation of menstruation in a female human, and typically occurs between the ages of about 45 and about 55. Menopause is marked by dramatic variations in hormone levels such as, for example, decreases in estrogen levels due to the ovaries no longer producing eggs.

“Spaying” or “neutering” as used herein means surgical procedures performed on animals for population control. “Neutering” is the removal of the testicles from a male animal, and “spaying” is the removal of ovaries from a female animal. “Spaying” or “neutering” can also cover pets that have been altered with respect to their sex organs, for example, through natural causes such as an anatomical defect, biochemical or genetic abnormality, or disease, for example, post-andropausal having reduced circulating estradiol levels relative to a healthy non-obese control animal.

“Treating” or “treatment” as used herein means the treatment of the conditions of overweight and obesity in a mammal and includes:

(i) inhibiting the conditions of overweight and obesity, i.e., arresting its development;
(ii) relieving the conditions of overweight and obesity, i.e., causing regression of the conditions of overweight or obesity; or
(iii) relieving the symptoms resulting from the conditions of overweight or obesity, i.e., relieving the conditions of overweight or obesity without addressing the underlying disease or condition.

“Preventing” as used herein means to stop the conditions of overweight and obesity from occurring.

“Ameliorating” as used herein means to make the conditions of overweight and obesity become bearable.

“Healthy body weight” as used herein means body weight range that is deemed to be healthy. It can be determined by Body Mass Index (BMI) based on height and weight. According to the U.S. CDC guidance, the range of BMI for a normal body weight is 18.5 kg/m2 to 24.9 kg/m2.

“Healthy body fat” as used herein means age-adjusted percentage of body fat that is considered healthy for a mammal. Healthy body fat comprises: (i) essential body fat, and (ii) storage body fat. Essential body fat is required for maintaining life's necessary functions and reproduction. Storage body fat consists of fat accumulation in adipose tissue. The present invention intends to limit the percentage of the storage body fat to minimize 5 the adverse weight gain.

“Fat stem cells” or “adipose stem cells” as used herein means multipotent cells that can differentiate into tissue cells, such as, adipocytes (i.e., fat cells). They are ubiquitously available and are found in adipose tissue. Fat stem cells are also known as “adipose-derived adult stem (ADAS) cells”, “adipose-derived stromal cells (ADSC)”, “adipose stromal cells (ASC)”, “adipose mesenchymal stem cells (AdMSC)”, “preadipocytes”, “processed lipoaspirate (PLA) cells”, and “adipose-derived stromal/stem cells (ASCs)”.

“Adipocytes” as used herein means the cells that specialize in storing energy as fat for the body and are the primary cells that compose adipose tissue. “Adipocytes” are also known as “lipocytes” and “fat cells”.

“Adipose Tissue” as used herein means the connective tissue comprising adipocytes (i.e., fat cells). Adipose tissue is generally where the body deposits and stores excess fat. Adipose tissue encompasses, without limitation, white, brown, and yellow adipose tissue.

“Body's fat storage capacity” as used herein means the body's ability to store energy as fat for the body in adipose tissue. The body fat's storage capacity increases with increasing numbers of adipocytes.

Referenced herein are trade names for components including various ingredients utilized in embodiments of the invention. The inventors herein do not intend to be limited by materials under a certain trade name. Equivalent materials (e.g., those obtained from a different source under a different name or reference number) to those referenced by trade name may be substituted and utilized in the descriptions herein.

Compositions Comprising a Mannoheptulose (mHep)

Specifically, the present invention provides for a composition comprising an effective amount of a mHep for treating overweight and obesity in a mammal. In one embodiment, the compositions of the present invention are food compositions intended for oral ingestion by the mammal, preferably a human or a pet (e.g., cats, dogs). In certain aspects, the compositions are intended for consumption by a pet, wherein the compositions are selected from the group consisting of a pet food, a supplement, and a liquid.

“Pet food” is a composition intended to be ingested by a pet, and preferably a domesticated pet such as, for example, a cat, a kitten, a puppy, or a dog. As used here “pet food” is intended to include things like treats (e.g., biscuits), chews, raw hides, and the like. In an embodiment, the “pet food” is designed specifically for a growing pet (e.g., puppy food) or In another embodiment, the “pet food” is a “complete and nutritionally balanced pet food”. This means that it contains all known required nutrients in appropriate amounts and proportions based on recommendations of recognized authorities in the field of pet nutrition, and is therefore capable of serving as a sole source of dietary intake to maintain life or promote production, without the addition of supplemental nutritional sources. For an example of a “nutritionally balanced” dog food, see the guidelines in the National Research Council, 1985, Nutritional Requirements for Dogs, National Academy Press, Washington D.C., or Association of American Feed Control Officials, Official Publication 1996).

In another embodiment, the pet food composition may be a dry composition (e.g., kibble, and having a total moisture content of from about 0% to about 16%, by weight of the product), a moist or a semi-moist composition (i.e., having a total moisture content of from about 16% to about 50%, by weight of the product), a wet composition (i.e., having a total moisture content of greater than about 50%, by weight of the product), or any mixture thereof.

In yet another embodiment, the pet food composition may be a “high fat food”, preferably a “high fat pet food”, comprising a fat content present in the pet food composition, on a dry matter basis: a) of at least about 15% by weight of the composition; b) of at least about 25% by weight of the composition; c) of at least about 35% by weight of the composition; or d) of at least about 45% by weight of the composition. It is a common practice to intentionally add some fat to pet food compositions to improve palatability. However, when the levels of fat begin to exceed 10% of the pet food composition, then the pet food is considered “high fat” by the consumers. High fat pet food is hard to market given the general consumers preference for lowfat alternatives that will deliver the right balance of nutrition to their pets.

Without wishing to be bound by theory, for example, with high fat pet food composition, it is believed that by adding an effective amount of a mHep into the high fat pet food composition, the mHep interacts with the adipocytes to negate the negative effects of the fat. Higher concentrations of mHep can be added to increasing concentrations of fat. to a level where the presence of the high fat content in the pet food composition has minimal or nil impact on the body fat accumulation of the animal consuming the food. In an embodiment, it is believed that the mHep is believed to be effective at mitigating the negative effects, even at very high levels of fat content, for example, to at least about 45% by weight of the composition.

Embodiments related to compositions, preferably pet food compositions, of the present invention may further comprise a source of carbohydrate. In one embodiment, the compositions may comprise from about 35%, by weight of the composition, up to about 50%, 5 by weight of the composition, carbohydrate source. In other embodiments, the composition can comprise a source of carbohydrate at from about 35% to about 45%, by weight of the composition, or from about 40% to 50%, by weight of the composition. Non-limiting examples of carbohydrate source include, grains or cereals such as rice, corn, milo, sorghum, barley, wheat, and the like are illustrative sources of carbohydrate.

The compositions, preferably pet food compositions, of the present invention may also contain other materials such as, but not limited to, dried whey and other dairy by-products, beet pulp, cellulose, fiber, fish oil, flax, vitamins, minerals, flavors, antioxidants, and taurine. Optionally, other beneficial ingredients may be present in the compositions, preferably pet food compositions, of the present invention, for example, crude protein as a percentage of the composition in the range of from about 10% to about 90% by weight of the composition, from about 20% to about 50% by weight of the composition, from about 20% to about 40% by weight of the composition, or from about 20% to about 35% by weight of the composition. Other optional ingredients in the pet food composition may include: probiotic components (Bifidobacteria and/or Lactobacillus), prebiotic components (e.g., fructoooligosaccharides, PCT Publication No. WO2005/0158294, for example), omega-6 and omega-3 fatty acids, carnitine, hexametaphosphate, glucosamine, chondroitin sulfate, carotenoids including beta carotene, vitamin E, and lutein.

“Supplement” as used herein is intended to cover both “dietary supplement” and “food supplement” and means a product intended to be ingested separately in addition to the normal diet of the mammal, or added to a food, preferably a pet food, or added to a liquid intended for consumption by the animal.

“Liquid” as used herein means fillers, gravies, sauces, beverages, supplemental water, and combinations thereof. The liquid is intended to be ingested separately in addition to the normal diet of the mammal, or added to a food, preferably a pet food.

Mannoheptulose (mHep)

The compositions of the present invention comprises an effective amount of an mHep for treating or preventing overweight and obesity in a mammal to which the composition has been administered. Where the composition is preferably a pet food, the amount of mHep as a percentage of the composition is in the range of from about 0.0001% to about 10% by weight of the composition, from about 0.1% to about 5% by weight of the composition, or from about 0.1% to about 3% by weight of the composition, wherein the weight of the composition is on a dry matter basis.

Although it should be noted that greater percentages of mHep can be supplied because it has a high therapeutic window unlike other caloric restriction mimetics, such as resveratrol, which has a high effective dose but strong cytotoxic (50-100 μm) making it to be more difficult to administer. In other various embodiments, the amount of mHep is about 1.0%, 1.5%, 2.0%, 2.5%, 3.0%, 3.5%, 4.0%, 4.5%, 4.0%, 4.5%, 5.0%, 5.5%, 6.0%, 6.5%, 7.0%, 7.5%, 8.0%, 8.5%, 9.0%, 9.5%, or 10.0% by weight of the composition.

When the composition is formulated as a dietary supplement, the composition may be formulated to contain mHep at higher concentrations (i.e., 2×, 3×, 4×, 5×, etc.) to be amenable for administration to a mammal in the form of a tablet, capsule, liquid concentrated, or other similar dosage form, or to be diluted before administrations, such as by dilution in water, spraying or sprinkling onto a composition such as, for example, a pet food, and other similar modes of administration.

The mHep used in the present invention can be derived from any suitable source. Preferred sources of mHep include:

i. a naturally-derived source of mHep;
ii. a synthetically-derived source of mHep;
iii. a microbial-derived source of mHep;
iv. a combination of a) and b);
v. a combination of a) and c);
vi. a combination of b) and c); or
vii. a combination of a), b) and c).
Naturally-Derived mHep

The mHep may be obtained from a “naturally-derived source”, such as, a component of plant matter such as an avocado, avocado extract, avocado meal, avocado concentrate, or other enriched source of mHep. Non-limiting examples of enriched sources of mHep include alfalfa, fig, or primrose. The plant matter may include the fruit, seed (or pit), branches, leaves, or any other portion of the relevant plant or combinations thereof.

Avocado (also commonly referred to as alligator pear, aguacate, or palta) contains unusually enriched sources of mHep, as well as related sugars and other carbohydrates. Avocado is a sub-tropical evergreen tree fruit, growing most successfully in areas of California, Florida, Hawaii, Guatemala, Mexico, the West Indies, South Africa, and Asia. Species of avocado include, for example, Persea Americana and 5 Persea nubigena, including all cultivars within these illustrative species. Cultivars may include ‘Anaheim,’ ‘Bacon,’ ‘Creamhart,’ ‘Duke,’ ‘Fuerte,’ ‘Ganter,’ ‘Gwen,’ ‘Hass,’ ‘Jim,’ ‘Lula,’ ‘Lyon,’ ‘Mexicola Grande,’ ‘Murrieta Green,’ ‘Nabal,’ ‘Pinkerton,’ ‘Queen,’ ‘Puebla,’ ‘Reed,’ ‘Rincon,’ ‘Ryan,’ ‘Spinks,’ opa Topa, ‘Whitsell,’ ‘Wurtz,’ and ‘Zutano.’ The fruit of the avocado is particularly preferred for use herein, which may contain the pit or wherein the pit is removed or at least partially removed. Fruit from Persea Americana is particularly preferred for use herein, as well as fruit from cultivars which produce larger fruits (e.g., about 12 ounces or more when the fruit is mature), such as Anaheim, Creamhart, Fuerte, Hass, Lula, Lyon, Murrieta Green, Nabal, Queen, Puebla, Reed, Ryan and Spinks.

Other examples of naturally-derived sources of mHep may include plant matter from alfalfa, fig, or primrose, which has been reported to provide relatively high levels of mHep.

Alfalfa is also referred to as Medicago sativa. Fig or Ficus carica (including Cluster fig or Sycamore fig, for example) may also be used, as well as primrose or Primula officinalis.

Synthetically-Derived mHep

The mHep may be obtained from a “synthetically-derived source”. Synthetic mHep is commercially available (Glycoteam GmbH (Hamburg, DE)) in highly purity forms. However, the highly purified synthetic mHep have the same activity level as the naturally-derived source of mHep, such as avocado extract.

Microbial Derived mHep

The mHep may be obtained from a “microbial derived source”. One skilled in the art will understand how to determine the appropriate amount of mHep to be added to a given composition. Non-limiting examples of some of the factors that may be taken into consideration include: (i) the type of composition (e.g., pet food composition vs. dietary supplement), (ii) the average consumption of specific types of compositions by different animals, and (iii) the manufacturing processes and conditions under which the composition is prepared. The compositions of the present invention can be made according to any method suitable in the art such as, for example, that described in Waltham Book of Dog and Cat Nutrition, Ed. ATB Edney, Chapter by A. Rainbird, entitled “A Balanced Diet” on pages 57 to 74, Pergamon Press Oxford.

In another aspect, the present invention is directed to a pet food composition comprising a mHep for promoting or maintaining a healthy body fat of growing pets, and wherein the mHep is present in the composition at from about 0.0001% to about 10% by weight of 5 the composition, from about 0.01% to about 5% by weight of the composition, or from about 0.1% to about 3% by weight of the composition. By the term “healthy body fat”, it is intended to mean age-adjusted percentage of body fat that is considered healthy for the mammal. For example, if the mammal is a human, one could refer to the World Health Organization (WHO) “Age-Adjusted Body Fat Percentage Recommendations” as a guideline. They have separate charts for men and women, as women tend to have higher percentage of body fat for reproductive reasons.

TABLE 1
Body Fat Percentage Recommendations

Women
Age Underfat Healthy
Range Overweight Obese
20-40 yrs Under
21% 21-33% 33-39% Over 39%
41-60 yrs Under
23% 23-35% 35-40% Over 40%
61-79 yrs Under
24% 24-36% 36-42% Over 42%
Men
Age Underfat Healthy
Range Overweight Obese
20-40 yrs Under
8% 8-19% 19-25% Over 25%
41-60 yrs Under
11% 11-22% 22-27% Over 27%
61-79 yrs Under
13% 13-25% 25-30% Over 30%

### Administration of the Compositions

The composition of the present invention can be administered to the mammal by a variety of routes of administration. Such routes include, for example: oral, intranasal, intravenous, intramuscular, intragastric, transpyloric, subcutaneous, rectal, and the like. In an embodiment, the preferred administration route is “oral” administration of the composition. “Oral” administration means that the mammal ingests the composition or a human feeds the mammal the composition of the present invention, and wherein the “human” that feeds the composition may be, for example, a pet owner, physician, veterinarian or other health professionals (e.g., pharmacists).

Feeding instructions that which informs and/or directs the human about the use and how to feed the composition to the mammal may include, for example, oral or written direction (e.g., from a physician, veterinarian, other healthy professional); radio, internet or elevision media (e.g., advertisement); and/or packaging associated with the composition (e.g., label present on the packaging). Additionally, administration in accordance with the present invention may be continuous or intermittent, and will vary depending upon the subject/patient. For example, upon review of the subject's/patient's physiological condition, and whether the purpose of the administration is therapeutic or prophylactic, and others factors that become known to the skilled practitioners.

### Methods of Using the Compositions

It has been discovered in accordance with the present invention that mHep inhibits the differentiation process of fat stem cells, specifically, mesenchymal stem cells (MSC), into adipocytes when cultured in the presence of differentiation medium. After culturing, the inventors discovered that the mHep treated fat stem cells have an overall gene expression pattern that is similar to the undifferentiated cells that were not given the differentiation medium. It has also been discovered that mHep effectively inhibit the ability of differentiating fat stem cells to accumulate lipid. These results indicate that mHep might be able to protect adipose tissue from biological changes induced by the excessive accumulation of body fat through reduction in the body's fat storage capacity.

In an aspect of the present invention, the present invention is directed to a method of treating or preventing overweight and obesity in a mammal, wherein the method comprises administering to the mammal a composition comprising an effective amount of a mHep. In an embodiment, the mammal is consuming more than the minimum caloric requirements or is not on a diet regimen.

In another embodiment, the mammal is a human (e.g., woman, man). When referring to a human, the terms “overweight” and “obesity” may be used to identify states of body weight/body fatness based on the human subject's Body Mass index (BMI). Table 2 below sets out ranges of BMI for each body state according to the guidelines provided by the U.S. CDC. BMI is determined by taking the subject's weight and dividing it by the height.

TABLE 2
BMI and Weight Status for Humans
BMI (kg/m²) Weight Status

Below 18.5 Underweight
18.5-24.9 Normal
25.0-29.9 Overweight
30.0 and above Obese

In another embodiment, the mammal is a pet selected from:

a) a growing pet selected from the group consisting of a reproductively-intact puppy, a reproductively-intact kitten, an overweight or obese puppy, an overweight or obese kitten, a spayed or neutered puppy, and a spayed or neutered kitten;
b) an adult pet selected from the group consisting of a reproductively-intact adult dog, a reproductively-intact adult cat, an overweight or obese adult dog, an overweight or obese adult cat, a spayed or neutered adult dog, a spayed or neutered adult cat; or
c) a senior pet selected from the group consisting of a reproductively-intact senior dog, a reproductively-intact senior cat, an overweight or obese senior dog, an overweight or obese senior cat, a spayed or neutered senior dog, and a spayed or neutered senior cat. When the mammal is a pet, the terms “overweight” and “obesity” refer to states of body weight/body fatness based on an assessment of the animal's body using the Body Condition Chart (BCS) for Assessing Obesity in Cats and Dogs, developed by Greg Sunvold from JAMS® (1998), as reproduced in FIGS. 1A (dogs) and 1B (cats). The chart provides 5 types of body weight/fatness status: (1) thin, (2) underweight, (3) ideal, (4) overweight, and (5) obese, with defining physical characteristics for each type to help with the assessment. The benefit of the chart approach is that it provides some uniformity to the assessment.

It has also been discovered that the degree of the adipocyte's response is dependent on the timing of the mHep administration relative to the stage of adipocyte differentiate, since mHep appears to have limited effect on adipocytes once they have differentiated and are accumulating lipids. Accordingly, the benefit of mHep on adipocyte functionality is dependent on early mHep administration prior to the initiation of adipocyte differentiation. Once fat stem cells have differentiated into adipocytes it is difficult to get rid of them. Therefore, the objective of early on administration of mHep to a mammal is to inhibit the differentiation process and limit your 25 body's reserve of adipocytes.

In another embodiment, the mammal is a growing pet such as, for example, a kitten or a puppy. The kitten may be a domesticated cat that is 3 years old or less, 2 years old or less, or 1 year old or less. The puppy may be a domesticated dog that is 3 years old or less, 2 years old or less, or 1 year old or less. Preferably, the growing pet is in need thereof such treatment since the animal is overweight or obese, or the method is a prophylactic to prevent the mammal from becoming overweight or obese. Non-limiting examples of a growing pet in need of such treatment include an overweight puppy, an obese puppy, an overweight kitten, or an obese kitten. The composition comprising mHep is administered to growing pets before they have fully developed to the normal adult weight, so it is not possible or healthy to suppress weight gain altogether. The method of the present invention allows for a net weight gain of lean body mass yet at the same time treats or prevents weight gain derived from excessive fat accumulation. Accordingly, for growing pets, the overweight or obesity being targeted by this new method is characterized by a weight gain derived substantially from excessive fat accumulation in adipose tissues. It is desirable that the composition of the present invention has a sparing effect on lean body mass.

In another embodiment, the growing pet may be reproductively-intact or preferably, may have been spayed or neutered. It is possible that after weaning but before reaching sexual maturity. The removal of sex organs results in markedly decreased levels of endogenous sex hormones (e.g., estrogen). Consequently, weight gain is common in mammals following spaying or neutering. Accordingly, the methods of the present invention would benefit growing pets that have been spayed or neutered.

In another embodiment, the method is for preventing overweight and obesity in a mammal having a body that is characterized as thin, underweight or ideal. It is clear that if the mammal is obese with appreciable levels of body fat storage, due to its mechanism of action, mHep will not be able to treat that animal. However, it is believed that mHep would be beneficial to prevent overweight and obesity in adult/senior pets, if they have not developed appreciable levels of body fat. In this scenario, MH would help to prevent/minimize the “middle_age spread” and “age_related increase in body fat” leading to overweight and obesity in an otherwise lean adult/senior pet, irrespective of whether they are reproductively-intact or neutered.

Of this embodiment, the mammal that would be suitable for this preventative method are ones having percentages of total body fat between from about 0.1% to about 20%, or from about 0.5% to about 15%, or from about 1% to about 10%, of the total body weight. Alternatively, another way of identifying if a mammal is overweight or obese is by checking if the mammal has (i) a body condition characterized as thin, underweight, or ideal, as determined based on a body condition chart as disclosed herein; or (ii) a body condition score (BCS) of 1 to 3 based on a scale from 1 to 5, wherein the BCS is deteremined according to the Body Condition Score Test asdisclosed herein.

For example, FIGS. 1A & 1B provide the body condition chart for dogs and cats (available from IAMS® (1998)). The body condition chart lists 5 body conditions (thin, underweight, ideal, overweight and obese) along with physical criteria that may 5 be easily viewed and measurable by anyone such as, for example, non-professionals (e.g., pet owner), and professionals (e.g., veterinarian and animal practiconer).

The BCS method is the most accessible and commonly used method for estimating obesity in a pet. Under the BCS method, physical examination, visual observation, and palpation may be used to assign a body condition score. The body condition score is a semi-quantitative assessment of body fat with the following scale 1 to 5 by 0.25 points; 1=emaciated, 2=thin, 3=average, 4=fat, and 5=obese. In another embodiment, wherein the method administration to the mammal of a composition comprising an effective amount of an mHep inhibits or reduces the differentiation process of fat stem cells into adipocytes in the mammal. In the present invention, “an effective amount” of a mHep refers to the amount of mHep in a composition of the present invention, when administered to a mammal, preferably a human or a domesticated animal such as, for example, a pet or companion animal (e.g., cats, dogs) sufficient to effect treatment or prevention of overweight and obesity. This “effective amount” of a mHep, which is a component of the composition of the present invention, will vary depending upon the condition and its severity, the manner/route of administration, the age of the mammal to be treated and its overall health, but can determined routinely by one of ordinary skill in the art having regard to his own knowledge and to this disclosure.

The dose of the mHep will depend upon the form in which the mHep is delivered to the mammal. The dose will vary depending on the size and condition of the pet to which mHep is to be administered. Dosage in the range of from about 0.0001 or about 0.001 g/kg to about 1 g/kg can be beneficial in some embodiments. As used herein, when dosage in mg/kg is used, the mg refers to the level of the mHep and kg refers to kilograms of body weight of the mammal, preferably where the mammal is a human or pet such as, for example, a cat or dog. In one embodiment, the dosage of the component provided to a pet on a daily basis may be from about 0.1, 0.5, 1, 2, or 5 mg/kg to about 15, 20, 50, 100, 150, or 200 mg/kg, and all combinations of these ranges, wherein “mg” refers to the level of the mHep and “kg” refers to kilograms of body weight of the mammal. In one embodiment, the daily dosage to the pet may be from about 0.1 mg/kg to about 50 mg/kg, from about 1 mg/kg to about 20 mg/kg, 1 mg/kg to about 15 mg/kg, from about 2 mg/kg to about 10 mg/kg, or from about 2 mg/kg to about 5 mg/kg, or about 2 mg/kg. In certain embodiments, these amounts may translate to compositions comprising less than about 5%, or less than about 2%, or from about 0.0001% to about 0.5%, or from about 0.1% to about 10%, or from about 5%, of mHep, all by weight of the composition. In certain embodiments, the mHep is present in the compositions in the range of from about 0.0001% to about 10%, or from about 0.01% to about 5%, or from about 0.1% to about 3%, by weight of the compositions. All ranges therebetween are envisioned. The level of mHep may be determined by one of ordinary skill in the art based on a variety of factors, for example, the form of the composition. For example, when the composition is a pet food composition it is selected from a dry composition, a semi-moist composition, wet composition, or supplement, or any other form or mixture thereof. The ordinarily skilled artisan will be able to utilize the preferred dosage and determine the optimal level of mHep within a given composition.

In another embodiment, wherein the administration of the mHep results in the inhibition or reduction of the differentiation process of fat stem cells into adipocytes results in lower levels of adipocytes such that the mammal's body's fat storage capacity decreases by about at least 5%, about at 10%, or about at least 20%, relative to an untreated mammal.

In another aspect, the present invention is directed to a method to promote or maintain a healthy body weight in a mammal, wherein the method comprises administering to the mammal an effective amount of a composition comprising a mHep to prevent or reduce excessive body fat in the mammal. “Excessive body fat” may be any amount that qualifies the mammal to be considered overweight or obese.

In yet another aspect, the present invention is directed to a method for preventing or ameliorating overweight and obesity in a mammal comprising feeding to the mammal a composition comprising a mHep present in the composition. The mHep is present at from about 25 0.0001% to about 10% by weight of the composition, from about 0.01% to about 5% by weight of the composition, or from about 0.1% to about 3% by weight of the composition. The composition is selected from the group consisting of a pet food, a supplement and a liquid, wherein the supplement or the liquid can be taken separately or added to the pet food, and wherein the mammal is an adult pet or a senior pet.

In yet another aspect, the present invention is directed to a method for preventing or ameliorating overweight and obesity in a mammal comprising feeding to the mammal a composition comprising a mHep. The mHep is present in the composition at from about 0.0001% to about 10% by weight of the composition, from about 0.01% to about 5% by weight of the composition, or from about 0.1% to about 3% by weight of the composition, wherein the composition is selected from the group consisting of a pet food, a supplement and a liquid, wherein the supplement or the liquid can be taken separately or added to the pet food, and wherein the mammal is an adult pet or a senior pet.

In yet another aspect, the present invention is directed to a method for increasing 5 lifespan of a mammal, comprising administering to the mammal a composition comprising a daily dosage amount of 1 mg/kg to about 50 mg/kg, or 1 mg/kg to about 15 mg/kg, or 1 mg/kg to about 5 mg/kg, of a mHep. It is expected that mammals that are exposed to longer duration of the mHep, for example, by starting with the treatment shortly after birth, will reap more benefits in terms of increased lifespan. In an embodiment, the administration starts when the mammal is about 1 month old or less, about 6 months old or less, about 1 year old or less, about 2 years old or less, or about 3 years old or less.

In an embodiment, the reduction of caloric intake by the mammal is at least about 5%, at least about 10%, at least about 20%, at least about 30%, at least about 40%, or at least about 15 50%. In another embodiment, the lifespan increases by about 10% or higher, about 15% or higher, about 20% or higher, about 25%, about 30% or higher, or about 35% or higher.

In another embodiment, the method of increasing lifespan wherein:

(i) the administration of mHep begins when the mammal is about 1 month old and the lifespan increase is about 30% or higher;
20 (ii) the administration of mHep begins when the mammal is about 6 months old and the lifespan increase is about 25% or higher;
(iii) the administration of mHep begins when the mammal is about 1 year old and the lifespan increase is about 20% or higher;
(iv) the administration of mHep begins when the mammal is about 2 years old and the lifespan increase is about 15% or higher; and
(v) the administration of mHep begins when the mammal is about 3 years old and the lifespan increase is about 10% or higher.

### Combination Therapy

The composition comprising an effective amount of mHep or an effective amount of mHep of the present invention may be usefully combined with one or more other therapeutic agent or as any combinations thereof, in the treatment of overweight or obesity.

### Packages

The present invention also covers an article of commerce, preferably in form of a package, containing a composition comprising an effective amount of a mHep as described hereinabove together with a feeding manual with instructions for how to orally administer composition to the feline companion animal. Any standard packaging that is suitable for delivery and sale of the compositions as disclosed herein can be used. The package can also include specific written benefit statements related to the treatment or prevention of overweight or obesity in a mammal, preferably where the mammal is a pet, and more preferably where the pet is a growing pet such as, for example, kitten or puppy.

The benefit statements can also relate to the health benefits resulted from such treatment or prevention, such as decrease body weight, healthy body weight, healthy body fat, and prolonged life span.

### EXAMPLES

The following examples are provided to illustrate embodiments of the invention and are not intended to limit the scope thereof in any manner.

### Example 1

### Preparation of Avocado Extract

Avocado extract containing enhanced levels of mannoheptulose is prepared in accordance with the following optional process, and utilized in compositions of embodiments of the invention.

Whole avocado fruit (about 900 kg) is provided. The fruit is split and the pits are removed, either partially or wholly, providing about 225 kg of pitted avocado halves. The raw avocado is charged to a disintegrator, whereupon some agitation, water (about 3000 kg) and CELLUBRIX (commercially available from Novozymes A/S) (about 1 liter) is further charged. The mixture is further agitated and concurrently heated to about 66° C. Upon completion of the charge, further CELLUBRIX (about 1 L) is added, and the entire mixture is CELLUBRIX held under agitation for about 12 hours at a controlled pH of about 5.5. The temperature is then further increased to about 80° C. and then held for at least about 2 hours. The resulting digested plant mixture is then filtered at 80° C. to provide the carbohydrate extract as the filtrate. The carbohydrate extract is then evaporated in a simplified recirculation system at 80° C., under vacuum, to provide the carbohydrate extract having from about 10% to about 20% solids and a pH of about 5.5. The extract is then further concentrated using a refractance window dryer to provide about 100 kg of the extract as a crystalline or powder (a yield of about 11% carbohydrate extract, based on the starting mass of the whole avocado fruit, which is analyzed as a yield from about 0.25% to about 4.5% mHep, based on the starting mass of the whole avocado fruit). It should be noted the amount of mHep found in avocados varies with the particular strain and state of ripeness of the fruit. The extract may be used in the compositions of embodiments of the invention.

### Example 2

### Effect of mHep on Mesenchymal Stem Cells (MSC) Differentiation

Study 1—This study describes an in vitro assay for testing the impact of mHep on the terminal differentiation of Mesenchymal Stem Cells (MSC) (i.e., fat stem cells) into functional adipocytes. MSC (C57BL/6) (Gibco) is cultured in adipocyte differentiating medium (Invitrogen) in the presence of palmitate at 37° C., with 5% CO2, to induce MSC differentiation from preadipocytesinto functional adipocytes. A control plate of MSC is cultured in Basal Medium without ADP (BP) as a negative control (i.e., reflect undifferentiated MSC).

The following samples are added to the MSC cultures with ADP to assess their effect on adipocyte differentiation: (i) mHep at 3 concentrations of 1.16 nM, 11 mM and 22 mM; (ii) whole-fruit avocado extract added at 0.2% with 25% mHep enrichment; and (iii) Resveratrol at 50 μM. Endpoint measures include: (i) oil red staining as a measure of accumulated fat; (ii) visual inspection of adipocytes; and (iii) an ATP assay to measure cell viability. Assay sensitivity allows for the testing of millimolar levels of mHep on adipocyte differentiation.

### Results 1

Results indicate the addition of palmitate in the differentiation cocktail increases lipid accumulation over the basal medium. MSC cultures with mHep (11 mM and 22 mM) or whole20 fruit avocado extract (0.2%) present in the medium reverses lipid accumulation to near basal levels. These results imply mHep inhibits fat cell differentiation and the ability of differentiated adipocytes to accumulate lipid based on oil red staining (FIG. 2). Results are supported by the reduced effectiveness of a lower mHep dose (1.16 mM) or a heat-inactivated source of wholefruit avocado extract (0.2%) to prevent fat cell differentiation and oil accumulation.

Study 2—In this study the impact of delaying the administration of mHep on MSC differentiation. 22 mM mHep was added to MSC cultures at 0, 4 and 7 days, and end-point measurements were taken with oil red staining as a measure of accumulated fat. A control is cultured in Basal Medium without ADP (BP) as a negative control (i.e., reflects undifferentiated MSC).

### Results 2

The inhibitory effects of mHep appear to be lost if mHep addition occurs after differentiation and the adipocytes are accumulating lipid. The following table shows the impact on MSC differentiation due to delay of mHep (22 mM) addition until days 4 or 7. The results in similar oil red-staining as MSC-derived control cells grown in differentiation media devoid of MH. In contrast, MH addition at day 0 results in similar inhibitory effects on lipid accumulation as noted previously. These results imply MH directly impacts the differentiation process of adipocytes to prevent lipid accumulation. MH appears to have no effect on adipocytes once they are differentiated and accumulating lipid. As such, any benefit of MH on adipocyte functionality is dependent on early MH administration prior to the initiation of adipocyte differentiation.

TABLE 3
MH (22 mM) addition
to differentiation media_
Percentage of oil red staining in MH-treated
cells vs. differentiated fat cells (±SD)_
Day 0_ 58% (±16%)_
Day 4_ 97% (±25%)_
Day 7_ 87% (±11%)_

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified. All documents cited in the Detailed Description of the Invention are, in relevant part, incorporated herein by reference; the citation of any document is not to be construed as an admission that it is prior art with respect to the present invention.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean 25 “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of treating or preventing overweight and obesity in a mammal, wherein the method comprises administering to the mammal a composition comprising an effective amount of a mannoheptulose (mHep).

2. The method of claim 1, wherein the method is for preventing overweight and obesity in a mammal having:
i. a body condition characterized as thin, underweight or ideal as determined based on a body condition chart as disclosed herein; or
ii. a body condition score (BCS) of 1 to 3 based on a scale from 1 to 5.

3. The method of claim 1, wherein the mammal is:
a) a growing pet selected from the group consisting of a reproductively-intact growing pet, an overweight or obese growing pet, and a spayed or neutered growing pet;
b) an adult pet selected from the group consisting of a reproductively-intact adult pet, an overweight or obese adult pet, and a spayed or neutered adult pet; or
c) a senior pet selected from the group consisting of a reproductively-intact senior pet, an overweight or obese senior pet, or a spayed or neutered senior pet.

4. The method of claim 3, wherein:
a) the adult pet is selected from the group consisting of a reproductively-intact adult dog, a reproductively-intact adult cat, a spayed or neutered adult dog, a spayed or neutered adult cat; or
b) the senior pet is selected from the group consisting of a reproductively-intact senior dog, a reproductively-intact senior cat, a spayed or neutered senior dog, and a spayed or neutered senior cat;
wherein the adult pet's or the senior pet's percentage of total body fat is between from about 0.1% to about 20%, or from about 0.5% to about 15%, or from about 1% to about 10%, of the total body weight.

5. The method of claim 3, wherein the mammal treated for overweight and obesity is a growing pet, wherein the overweight or obesity is characterized by a weight gain derived substantially from excessive fat accumulation in adipose tissues.

6. The method of any preceding claims, wherein the administration to the mammal of a composition comprising an effective amount of an mHep inhibits or reduces the differentiation process of fat stem cells into adipocytes in the mammal.

7. The method of claim 6, wherein the inhibition or reduction of the differentiation process of fat stem cells into adipocytes results in lower levels of adipocytes such that the mammal's body's fat storage capacity decreases by about at least 5% relative to an untreated mammal.

8. The method of claim 6, wherein the administration comprises feeding to the mammal a composition comprising an effective amount of a mHep, wherein the effective amount is a daily dosage amount of from about 0.1 mg/kg to about 50 mg/kg of the mHep, and wherein the composition is selected from the group consisting of a pet food, a supplement and a liquid, wherein the supplement or the liquid can be taken separately or added to the pet food.

9. The method of claim 1, wherein the mHep is derived from:
a) a naturally-derived source of mHep;
b) a synthetically-derived source of mHep;
c) a microbial-derived source of mHep;
d) a combination of a) and b);
e) a combination of a) and c);
f) a combination of b) and c); or
g) a combination of a), b) and c).

10. The method of claim 9, wherein the naturally-derived source of mHep is selected from the group consisting of avocado, avocado extract, avocado meal, avocado concentrate, avocado juice, alfalfa, alfalfa extract, alfalfa meal, alfalfa concentrate, alfalfa juice, fig, fig extract, fig meal, fig concentrate, fig juice, primrose, primrose extract, primrose meal, primrose concentrate, primrose juice, and mixtures thereof.

11. The method of claim 1, wherein the mHep is present in the composition at from about 0.0001% to about 10% by weight of the composition, from about 0.01% to about 5% by weight of the composition, or from about 0.1% to about 3%, by weight of the composition.

12. The method of claim 1, wherein the composition is a high fat food having a fat content present in the composition:
a) of at least about 10% by weight of the composition;
b) of at least about 15% by weight of the composition;
c) of at least about 25% by weight of the composition;
d) of at least about 35% by weight of the composition; or
e) of at least about 40% by weight of the composition.

13. A pet food composition comprising mannoheptulose (mHep) for promoting or maintaining a healthy body fat of growing pets, and wherein the mHep is present in the composition at from about 0.1% to about 3% by weight of the composition.

14. The composition of claim 13, wherein the composition is a high fat food having a fat content of at least about 15% by weight of the composition.

15. The composition of claim 13, wherein the mHep is naturally derived.

16. The composition of claim 15, wherein the naturally-derived source of mHep is selected from the group consisting of avocado, avocado extract, avocado meal, avocado concentrate, avocado juice, alfalfa, alfalfa extract, alfalfa meal, alfalfa concentrate, alfalfa juice, fig, fig extract, figmeal, fig concentrate, fig juice, primrose, primrose extract, primrose meal, primrose concentrate, primrose juice, and mixtures thereof.

17. The composition of claim 13, wherein the composition is a nutritionally balanced pet food.

18. The composition of claim 17, wherein the nutritionally balanced pet food is a food for an pet selected from the group consisting of a reproductively-intact puppy, a reproductively-intact kitten, an overweight or obese puppy, an overweight or obese kitten, a spayed or neutered puppy, and a spayed or neutered kitten.

19. The composition of claim 13, wherein the composition is a pet treat.

20. The composition of claim 13, wherein the composition is a pet supplement.
